(19) United States
(12) Patent Application Publication
(10) Pub. No.: US 2013/0236570 A1
(43) Pub. Date: Sep. 12, 2013

(54) Use of hypertonic saline to draw fluid out of swollen tissue and relieve nasal congestion

(71) Applicant: Matthew Lutin, (US)

(72) Inventor: Matthew Lutin, (US)

(21) Appl. No.: 13/385,835

(22) Filed: Mar. 9, 2012

(51) Int. Cl. A61K 33/14; A61P 11/12

(52) U.S. Cl. 424/663

(57) ABSTRACT

The optimum concentration of salt in solutions to combat congestion of the mucosa is a relatively narrow range. This use of in hypertonic saline solutions for treatment of congestion of the mucosa having a concentration within the range of 2.3% to 2.7% w/v sodium chloride/water with the most preferred concentration being 2.4% to 2.6% w/v of sodium chloride in water brings maximum relief of congestion without causing irritation or discomfort.

### FIELD AND BACKGROUND OF THE INVENTION

This application relates to relief of discomfort arising from congestion whether due to infection, allergic response or simple mechanical irritation. While the use of hypertonic solutions to relieve nasopharyngeal congestion has been known, the usual concentration on the market contains approximately 3% sodium chloride. Hypertonic saline solutions containing 2% to 3% w/v sodium chloride/water are those more commonly known in the art. (Common salines on the market are 0.65%, 0.9%, and 3% sodium chloride.) However, it has now been found that the range at which hypertonic saline will provide most effective response without a rebound congestion is quite narrow, being from 2.3% to 2.7% w/v sodium chloride/water with the most preferred concentration being 2.4% to 2.6% w/v of sodium chloride in water. The method of the invention does not require use of a nasal cup or other complicated equipment.

Pain and discomfort are experienced when the mucous membrane swells as it becomes inflamed, blocking the drainage of fluid from the sinuses into the nose and throat. Mucus and fluid build up inside the sinuses, causing pressure and pain. The discomfort and problems related to treatment are further complicated because bacteria are more likely to grow in sinuses that are unable to drain properly. Bacterial infection then further increases inflammation and pain.

U.S. Pat. No. 6,645,532 to Lutin teaches use of hypertonic saline for preventing discomfort in the ears using hypertonic solution in a most preferred range of 2.4% to 2.6% w/v sodium chloride/water. The method of that patent requires that the individual retain the solution for at least 5 seconds, then expel the saline under pressure to avoid or remedy discomfort arising from obstruction/congestion in the eustachian tubes. The instant invention for relief of nasal congestion does not require expelling the saline under pressure, since there is no need to open the eustachian tubes for relief of simple nasal congestion.

U.S. Pat. No. 6,899,903 to Quillin teaches a composition for cleansing the sinuses using a solution containing Baptista tincturia colloidal silver, grapefruit seed extract and oregano juice and, further, incorporating 0.01% to 5% sodium chloride. The lone example uses a solution containing 0.75% sodium chloride. However, as indicated below, some of these concentrations of sodium chloride in water are ineffective and some cause undue irritation. The lower concentrations of sodium chloride in the compositions tested were essentially ineffective for purposes of relieving congestion.

U.S. Pat. No. 6,258,372 to Jones teaches use of a solution containing xylitol/xylose and between 0.45% and 0.95% sodium chloride with a preferred concentration of 0.65% sodium chloride, a concentration which is far less effective for relieving congestion than the preferred concentration disclosed and claimed herein.

U.S. Pat. No. 5,897,872 to Picciano teaches a nasal moisturizing saline solution containing iodine and hypotonic 0.65% sodium chloride.

Rabago, et. Al., “Efficacy of Daily Hypertonic Saline Nasal Irrigation Among Patients with Sinusitis: A Randomized Controlled Trial”. Journal of Family Practice, December 2002, Vol. 51, No. 12 :1049-1055 teaches use of 2% hypertonic saline as a nasal irrigation for treatment of sinusitis. The method of Rabago, et al. involves use of a nasal cup. That concentration of sodium chloride has been shown to be less useful in the testing shown below when using the much simpler methods of the invention.

### SUMMARY OF THE INVENTION

It is the purpose of this invention to relieve discomfort arising from congestion of the mucous membranes of the nasal passages. While there are many references which teach use of saline to relieve congestion, it has now been found that a very narrow range of salt concentration provides optimal relief without discomfort. Hypertonic saline of 2.3% to 2.7% (preferably 2.4% to 2.6%) gives optimum results without causing irritation. When lower levels of salinity are used, the treatment is less effective. When washes with higher salinity are used, there is often irritation to the membrane and the initial effect may be countered with a rebound swelling. The methods and equipment used may vary. However, any means which delivers the saline to the membranes is acceptable.

### DETAILED DESCRIPTION OF THE INVENTION:

As indicated in the Background of the Invention, many references teach a wide range of salt concentrations of saline without any suggestion that the concentration used as described herein would have any improved benefit. Moreover, none teach the simply application of saline within the range taught herein as a means of providing relief from congestion of the mucous membrane. Unlike the method taught in U.S. Pat. No. 6,645,532 to Lutin for relief from discomfort in the ears, there is no need for the patient suffering from simple congestion of the mucous membranes to exert pressure by blocking the nose when expelling the saline. In fact, mere washing of the membranes with the saline provides relief. The saline may be administered in any way that causes the solution to contact the mucous membrane. For example, the saline may be delivered by drops, in a mist, by syringe, such as an ear syringe, or by pump. Pressure in administration is not required and pressure in expelling fluid is not necessary.

The objective in choosing a solution was to find the concentration at which there was least irritation with good relief of congestion. It was noted that at concentration above 2.7% there was an immediate relief of congestion. However, when higher concentrations of salt were used there appeared to be a rebound increase in congestion following immediate relief.

### Materials and Methods

Saline solutions of varying strengths were prepared. The solution was administered as a mist through the nose, then allowed to flow out of the nose.
The following shows the results obtained with various concentration of salt (w/v salt/water).
No irritation −
Some irritation +
Moderate irritation ++
Severe irritation +++
Effectiveness was determined by subjective relief of congestion

### Hypertonic Solutions

| | |
|---|---|
| 2.1% - No irritation − | Less effective |
| 2.2% - No irritation − | Less effective |
| 2.3% - No irritation − | Less effective |
| 2.4% - No irritation − | Most effective |
| 2.5% - No irritation − | Most effective |
| 2.6% - No irritation − | Most effective |
| 2.7% - Some irritation + | Most effective |
| 2.8% - Some irritation + | |
| 2.9% - Moderate irritation ++ | |
| 3.0% - Moderate irritation ++ | |
| 3.1% - Severe irritation +++ | |
| 3.2% - Severe irritation +++ | |
| 3.2% - Severe irritation +++ | |

Many causes lead to congestion. Colds commonly trigger congestion, but any factor that causes the mucous membrane to become inflamed may lead to sinusitis. Many people with allergies suffer congestion (allergic rhinitis). Congestion arising from allergies often leads to recurring and/or chronic inflammation of the mucous membranes which can give rise to sinus infections. In fact, any condition which blocks the nasal passages increases the risk of sinusitis.

Inflamed, congested mucous membranes can also give rise to snoring. The method of the invention often decreases snoring by flushing out germs, contaminants, and pollutants (pollen/dust/sand/soot/smoke, etc.) from the sinus tissue.

The use of the solutions in accord with the methods of the invention provide means of prophylaxing against congestion arising from exposure to dust, smoke and other environmental irritants. For example, cleaning the mucous membranes with preferred solutions taught herein after exposure to dust such as that encountered in mining, milling or farming can prevent congestion and infection of the mucous membranes. The methods of the invention are particularly useful for cleansing the mucous membranes following exposure to smoke.

The solutions may be applied to the mucous membranes by any means which causes the solution to contact the membranes, such as aerosol, dropper, pump, ear syringe. A relatively new means of application, the nasal mist pump, has increased both sinus tissue area exposure and the amount of liquid solution being dispensed, resulting in a most effective solution for reducing sinus congestion. In many instances the low pressure nasal mist pump is the best device for administration of the hypertonic solutions. Additionally, the nasal mist pump is a preferred method of administration, since it can be loaded with solution, sealed and sold with the solution protected from exposure to contaminants. It provides a means of thoroughly bathing the membranes in the solution.

When the solution is administered as a stream by syringe, it is best to have the head bent over a basin or sink so that the solution may be allowed to flow out through the nose and mouth. When using a pump, dropper or aerosol (most convenient ways for administration) the solution can simply flow into the nose and out again to cleanse and dehydrate the membranes contacted.

Unlike with the prior invention of Lutin to prevent discomfort in the ears, there is no need to hold the nose while applying pressure. In fact, such application of pressure against a closed nose would not be appropriate in treatment of children

The compositions of the invention need not contain a preservative. In fact, preservative free hypertonic saline at 2.3% to 2.7% w/v (preferably 2.4% to 2.6% w/v) salt in water is quite effective and has not presented any problem in use so long as the solution is free of contamination. It should be noted that in any case where hypersensitivity or outright allergy is a cause of congestion, it is preferred that the solution be as free of additives as possible.

1. A method of relieving congestion of nasal mucosa comprising the steps of contacting the mucosa with a hypertonic saline solution containing 2.3% to 2.7% w/v saline wherein the saline is administered by spray, drops or a mist to the membranes, then allowed to flow out of the nose or mouth without pressure.

2. The method of claim 1 wherein the solution contains sodium chloride concentration of 2.4% to 2.6% w/v.

3. The method of claim 1 wherein the solution is administered by drops into the nose.

4. The method of claim 1 wherein the solution is administered as a mist.

5. The method of claim 1 wherein the solution is administered by syringe.

6. The method of claim 1 wherein the solution is administered via a nasal mist pump.
